(19) United States
(12) Patent Application Publication
(10) Pub. No.: US 2025/0149171 A1
(43) Pub. Date: May 8, 2025

(54) ENHANCING PERFORMANCE OF DIAGNOSTIC MACHINE LEARNING MODELS THROUGH SELECTIVE DEFERRAL TO USERS

(71) Applicant: Google LLC, Mountain View, CA (US)

(72) Inventors: Jim Huibrecht Winkens, London (GB); Alan Prasana Karthikesalingam, London (GB); Krishnamurthy Dvijotham, Sunnyvale, CA (US); Ali Taylan Cemgil, London (GB); Sumedh Kedar Ghaisas, London (GB)

(21) Appl. No.: 18/834,165

(22) PCT Filed: Jan. 30, 2023
(86) PCT No.: PCT/US2023/011903

§ 371 Date: Jul. 29, 2024

Related U.S. Application Data
(60) Provisional application No. 63/304,509, filed on Jan. 28, 2022.

(51) Int. Cl. G16H 50/20; G06V 10/764; G16H 30/20

(52) U.S. Cl. CPC G16H 50/20; G06V 10/764; G16H 30/20

(57) ABSTRACT

Methods, systems, and apparatus, including computer programs encoded on computer storage media, for classifying data points using a deferral model that determines whether to classify the data point using an output of one or more diagnostic machine learning models or to defer the data point for classification by one or more users.

### CROSS-REFERENCE TO RELATED APPLICATIONS

This application claims the benefit of priority to U.S. Provisional Application Ser. No. 63/304,509, filed Jan. 28, 2022, the entirety of which is incorporated herein by reference.

### BACKGROUND

This specification relates to processing inputs using neural networks.

Neural networks are machine learning models that employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters.

### SUMMARY

This specification describes a prediction system implemented as computer programs on one or more computers in one or more locations that uses a deferral model to improve the performance of a set of one or more diagnostic machine learning models.

Particular embodiments of the subject matter described in this specification can be implemented so as to realize one or more of the following advantages.

Deep learning-based AI systems can achieve substantial accuracy in many applications, e.g., medical diagnostics and product defect recognition. However, such systems are not always reliable and can fail in cases that would be diagnosed accurately by users, i.e., can misclassify an input that would have been correctly classified by a knowledgeable user.

This unreliability impacts deployment in safety-critical areas where the potential for AI error often means that oversight is crucial. However, leveraging the increased speed with which an AI system can make a classification (relative to an expert user) and the improved accuracy of the AI system (relative to the expert user) for a large number of possible inputs is desirable.

One such safety-critical application is medical imaging analysis, where diagnostic AI systems have demonstrated expert performance but where AI models can make errors in cases that can be diagnosed accurately by clinicians. Optimal care requires deference to the diagnostic opinion, i.e., that of the model or that of the clinician, that is most likely correct, but this is challenging to predict because failure modes of AI systems have been difficult to characterize. That is, it is difficult to determine a priori whether a given input is of a type that can be more accurately classified by a clinician than by a trained diagnostic machine learning model.

This specification describes techniques for resolving these issues by using a deferral model that can learn to decide when to rely on a diagnostic AI model and when to defer to a user, e.g., a clinician or other expert. This results in a system that achieves superior classification performance relative to both AI-based systems alone and clinicians alone.

In practice, regulatory requirements, engineering, data-sharing or other considerations may require the diagnostic AI to be accessible only as a “locked model” that cannot be modified and, in some cases, that has an unknown set of parameters and architecture.

Given this constraint, the described deferral model is compatible with any pre-existing diagnostic AI model without requiring it to be retrained. The described system uses only confidence scores from one or more “locked” (pretrained) diagnostic AI models as inputs to a “deferral” model that decides whether to make a prediction using the diagnostic AI models or defer to a user; and can therefore be implemented as a wrapper around any diagnostic AI model or ensemble of multiple AI models. That is, the described system can be used to improve the performance of any set of one or more AI model(s) without needing to modify the model(s) in any way.

Moreover, the deferral model can be used to adapt the AI system from one domain to another without needing to re-train the underlying machine learning models, resulting in significant resource savings. In particular, the AI system may have been trained on input data points that have a first distribution but may then need to be deployed to classify downstream data points that are distributed differently. By using only a small tuning data set drawn from the distribution of the downstream data points, the described system can adapt the AI system to function well on the downstream data points without needing to re-train the underlying model(s).

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example classification system.

FIG. 2 illustrates the operation of the deferral model after the parameters of the deferral model have been determined.

FIG. 3 shows an example of the composition of the tuning data set.

FIG. 4 is a flow diagram of an example process for performing a training process to determine the parameters of the deferral model.

FIG. 5 is a flow diagram of an example process for determining the upper and lower bounds of the deferral region(s) using the tuning data set.

Like reference numbers and designations in the various drawings indicate like elements.

### DETAILED DESCRIPTION

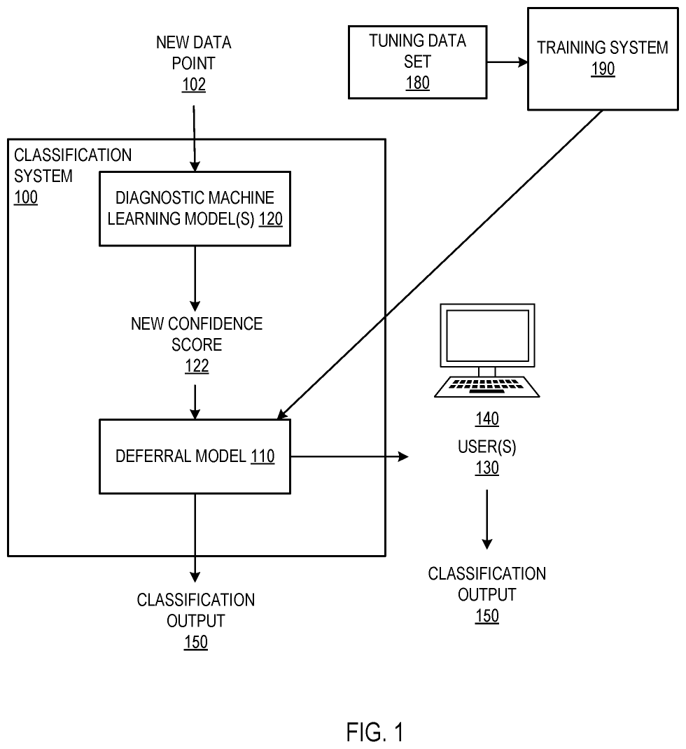

FIG. 1 shows an example classification system 100. The classification system 100 is an example of a system implemented as computer programs on one or more computers in one or more locations, in which the systems, components, and techniques described below can be implemented.

In particular, the system 100 receives a new data point 102 that needs to be evaluated to determine whether the new data point 102 has or does not have a particular property, i.e., evaluated to determine the likelihood that the new data point has the property. Thus, the system 100 is a classification system that classifies data points 102 as having or not having the particular property.

For example, each data point 102 can include one or more images (e.g. captured by an imaging device, such as a medical imaging device) and the particular property can be a property of a person or object depicted in the one or more images. Instead or in addition, the data point 102 can include text data or audio data characterizing an entity and the property can be a property of the entity characterized by the text or audio data.

Generally, the system 100 processes the new data point 102 to output a classification output 150 that indicates whether the new data point 102 has the property or not. For example, the classification output can be a binary indicator that is one value, e.g., zero, when the data point 102 is classified as not having the property and another value, e.g., one, when the data point 102 is classified as having the property. As another example, the classification output can be a first text string (e.g., “[property name] detected”) when the data point 102 is classified as having the property and another text string (e.g., “[property name] not detected”) when the data point 102 is classified as having the property.

As one example, each data point can include one or more diagnostic images of a corresponding patient, e.g., of all or a part of the body of the patient, and, optionally, additional information about the corresponding patient, e.g., information from the electronic medical record of the patient. In this example, the property can be a property that relates to the health of the corresponding patient.

For example, the one or more images can include a medical image generated by a medical imaging device; as particular examples, the image can be a computer tomography (CT) image, a magnetic resonance imaging (MRI) image, an ultrasound image, an X-ray image, a mammogram image, a fluoroscopy image, a fundus image, or a positron-emission tomography (PET) image.

As another example, the one or more images can include an RGB image generated by a camera sensor, e.g., of a mobile device or a digital camera.

For example, the property can indicate whether the patient has a particular medical condition, e.g., a particular type of cancer, e.g., breast cancer or lung cancer, hypertension, macular degeneration, diabetes, a skin condition, and so on.

As another example, the property can indicate whether the patient is at risk for suffering an adverse health event, e.g., a heart attack, a stroke, or an adverse kidney injury.

As another example, the one or more images can be images of an object manufactured at a manufacturing facility, e.g., an assembly line or other facility, and the property can indicate whether the object has a specified type of defect.

The system 100 processes the new data point 102 using a set of one or more trained diagnostic machine learning models 120, e.g., neural networks, to generate a new confidence score 122 for the new data point 102 that represents an estimated likelihood that the new data point has the particular property.

In some cases, the set includes multiple trained diagnostic models 120 and the new confidence score 122 is a weighted sum of individual confidence scores generated by the models 120 in the set. For example, the weights in the weighted sum can be equal for each diagnostic model 120 (and the new confidence score 122 can be the average of the individual confidence scores) or can be different for different models 120. More generally, the weights in the weighted sum can be determined by a training system that trained the models 120 and can be provided as input to the system 100.

In some other cases, the set includes only a single model 120 and the new confidence score 122 is the score generated by that single model.

Each diagnostic model 120 in the set can have any particular architecture that allows the model to process data items to generate a confidence score that represents a predicted likelihood that the data items have the particular property. For example, the models 120 can be convolutional neural networks or self-attention based neural networks, e.g., variants of a Vision Transformer, that are configured to classify sets of one or more input images.

Each model 120 in the set can have been trained on a set of training data using conventional machine learning techniques to optimize an objective function for the classification task, e.g., a cross-entropy loss or other appropriate objective function.

When there are multiple models 120 in the set, the different models 120 in the set can differ from one another in one or more ways. As one example, the different models 120 can have been trained on different subsets of a larger set of training data. As another example, the different models 120 can have been trained on the same training data, but in different orders. As yet another example, the different models 120 can have been trained starting from different initializations of the values of the parameters of the models 120.

More specifically, the system 100 can query the trained model(s) 120 to obtain confidence scores for provided inputs but does not require that the model(s) 120 be trained in any particular way and does not require access to the trained parameter values of the model(s) 120. In some implementations, the system 100 has access to the trained model(s) 120 only as a “locked model” that cannot be modified and, in some cases, that has an unknown set of parameters and architecture. For example, the system 100 can have access to an application programming interface (API) that allows the system 100 to query the trained model(s) 120 but that does not expose the underlying architecture of the models 120.

In other words, the system 100 can improve the performance of any of a variety of models 120 that have any of a variety of model architectures and that have been trained in any of a variety of ways. Thus, the described techniques are compatible with any model(s) 120 and do not require the model(s) 120 to be re-trained.

Instead of directly determining how to classify the new data point 102 from the new confidence score 122, the system 100 instead processes the new data point 102 using a deferral model 110 having parameters to determine whether to (i) classify the new data point 102 as having the particular property, (ii) classify the new data point 102 as not having the particular property, or (iii) provide the new data point 102 for presentation to one or more users 130 for evaluation of whether the given data point has the particular property.

That is, the deferral model 110 determines, using a set of parameters of the deferral model 110, whether the new confidence score 122 can be used to reliably classify the new data point 102. If so, the deferral model 110 uses the new confidence score to generate the classification output 150. If not, the deferral model 100 determines to provide the new data point 102 for presentation to a user 130 for evaluation instead of evaluating the new data point 110 using the new confidence score 122 generated by the model(s) 120.

In response to determining to provide the new data point 102 for presentation to the user 130, the system 100 or another system provides the new data point 102 for presentation the user 130, e.g., on a user device 140, and obtains, from the user 130, e.g., as a result of the user 130 submitting an input on the user device 140, an indication of whether the new data point 102 has the particular property or not. The system 100 then uses the indication to generate the classification output 150.

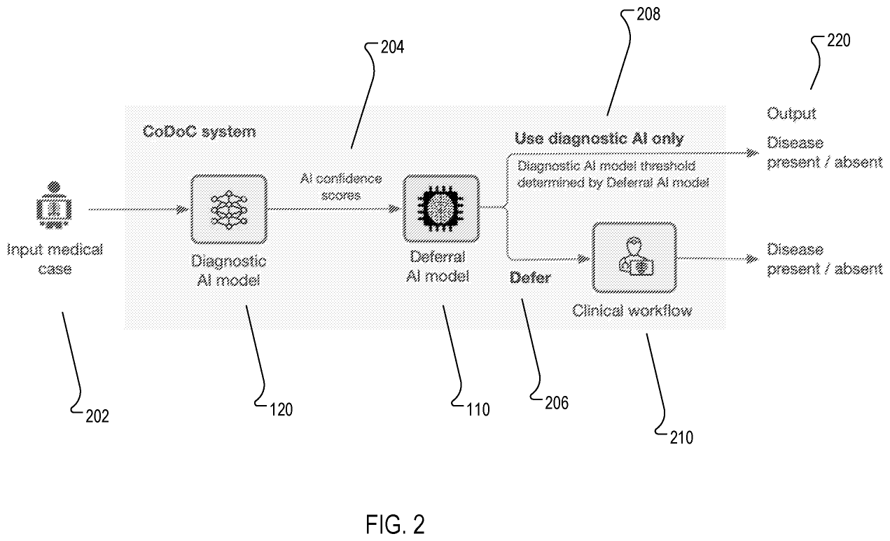

The parameters of the deferral model 110 and using the deferral model 110 are described in more detail below with reference to FIG. 2.

Prior to using the deferral model 110, a training system 190 determines (“learns”) the parameters of the deferral model 110 using a tuning data set 180.

The tuning data set 180 includes, for each of a set of tuning data points, (i) a confidence score generated by the set of trained diagnostic machine learning models for the tuning data point and (ii) a user indication for the tuning data point that identifies whether a user, e.g., the user 130, evaluated the tuning data point as having the particular property or as not having the particular property. More specifically, the tuning data points in the set include a plurality of positive tuning data points that have been labeled, e.g., by a user of the system 190 or as a result of testing performed on the person or object characterized by the tuning data point, as having the particular property and a plurality of negative data points that have been labeled as not having the particular property. For example, when the property is the presence of a medical condition, the label can be generated based on the results of diagnostic testing on the corresponding patient, e.g., a biopsy, an assay, and so on. When the property indicates whether the patient is at risk of suffering an adverse health event, the label can be generated based on the ground truth outcome, i.e., whether the corresponding patient actually suffered the adverse health event within some period of time of the data point being captured. When the property is the presence of a defect in a manufactured item, the label can be generated based on the results of diagnostic testing performed on the manufactured item.

Thus, each tuning data point has a respective “label” that is a ground truth indication of whether the tuning data point has the particular property or not.

The training system 190 determines the parameters of the deferral model 110 by optimizing an objective that is based on a specificity of the deferral model 110 on the tuning data set 180 and a sensitivity of the deferral model 110 on the tuning data set 180.

The specificity of the deferral model 110 measures the fraction of the positive tuning data points for which either (i) the deferral model 110 determined to classify the positive tuning data point as having the particular property or (ii) the deferral model 110 determined to provide the positive tuning data point for evaluation by a user and (iii) the user indication for the positive tuning data point identifies that the user evaluated the positive tuning data point as having the particular property.

The sensitivity of the deferral model 110 measures the fraction of the negative tuning data points for which either (i) the deferral model 110 determined to classify the negative tuning data point as not having the particular property or (ii) the deferral model 110 determined to provide the negative tuning data point for evaluation by a user and (iii) the user indication for the negative tuning data point identifies that the user evaluated the negative tuning data point as not having the particular property.

Generally, the training system 190 can determine the parameters based on the sensitivity and specificity without needing to have access to the parameter values of the diagnostic model(s) 120 or to further train the diagnostic model(s) 120. In particular, the only information relevant to the diagnostic model(s) that is required by the training system 190 in order determine the parameters are the confidence scores (that are generated by diagnostic model(s) after training) for the tuning data points in the tuning data set 180.

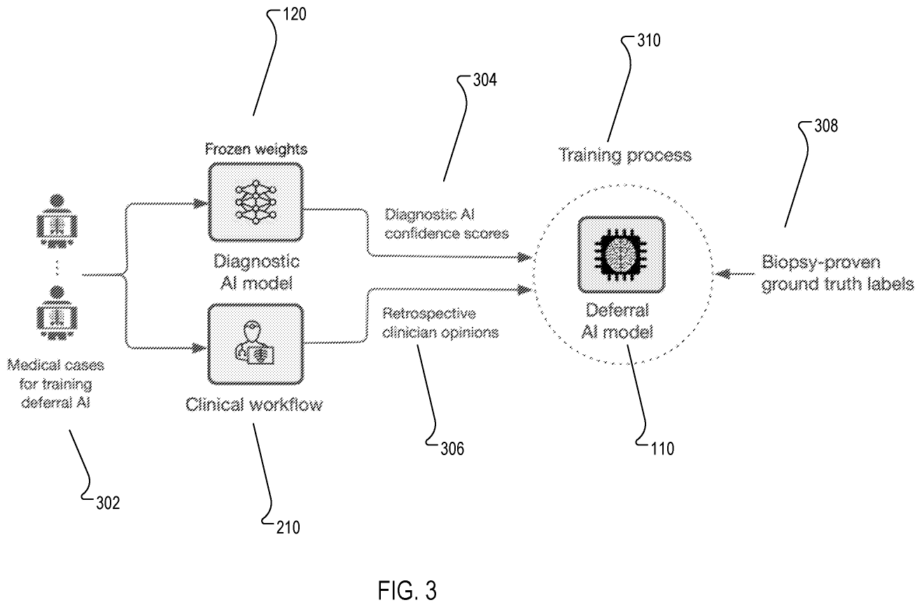

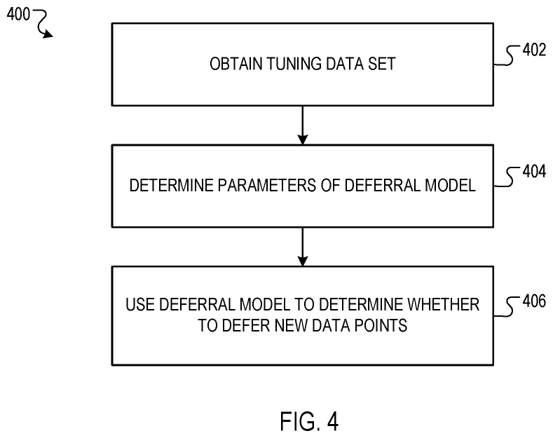

Determining the parameters is described in more detail below with reference to FIGS. 3 and 4.

FIG. 2 illustrates the operation of the deferral model 110 after the parameters of the deferral model 110 have been determined.

In the example of FIG. 2, the input data points are medical cases for corresponding patients, e.g., an input medical case 202, and the classification output indicates whether a specified disease is present or absent in the input medical case 202. A medical case is a data point that includes one or more medical images and, optionally, additional metadata about the corresponding patient, e.g., information from the patient's electronic health record.

As shown in FIG. 2, the system 100 processes the input medical case 202 using the diagnostic machine learning model(s) 120 (“diagnostic AI model”) to generate a confidence score 204 for the input medical case 204 as described above.

The system 100 processes the confidence score 204, e.g., a single confidence score or a weighted sum of a set of multiple confidence scores, using the deferral model 110 which determines whether to (i) defer 206 the medical case 202 to a clinical workflow 210 that involves evaluation of the input medical case 202 by one or more clinicians or (ii) use the diagnostic AI only 208 to classify the medical case 202 without involving the clinical workflow 210.

For example, as part of the clinical workflow 210, the system 100 or another system can present the medical case 202 to each of the one or more clinicians and can determine, from inputs received from the one or more clinicians, to classify the medical case as either indicating that the disease is present or that the disease is absent.

Thus, in response to determining to defer 206 the medical case 202, a classification 220 for the medical case 202 is generated using inputs received from one or more users, i.e., the one or more clinicians.

Alternatively, in response to determining to use the diagnostic AI only 208, the deferral model 110 generates the classification 220 for the medical case 202 using only the confidence score 204 generated by the model(s) 120.

Thus, when a classification for a new data point is required, the deferral model 110 receives as input a confidence score generated by the set of one or more trained diagnostic machine learning models 120 and determines, based on the parameters of the deferral model 110, whether to (i) classify the given data point as having the particular property, (ii) classify the given data point as not having the particular property, or (iii) provide the given data point for presentation to a user for evaluation of whether the given data point has the particular property.

In particular, the parameters of the deferral model 110 include respective lower and upper bounds for each of one or more deferral regions (i.e. sub-ranges within the range of possible values which the confidence score 122 can take), and an operating point (i.e. a value within the range of possible values which the confidence score 122 can take).

After training, the deferral model 110 determines to provide a given data point for presentation to a user for evaluation, e.g., to defer 206 the medical case 202, when the confidence score for the given data point falls within any of the one or more deferral regions. A score falls within a deferral region when the score is greater than or equal to the lower bound for the region and less than or equal to the upper bound for the region.

The deferral model 110 determines to use the confidence score to classify the given data point when the confidence score for the given data point does not fall within any of the deferral regions, e.g., to use diagnostic AI only 208 to classify the medical 202 using the classification score 204.

More specifically, the deferral model determines to (i) classify the given data point as having the particular property when the confidence score 122 satisfies the operating point (referred to as a “diagnostic AI model threshold” in FIG. 2) and is not within any of the one or more deferral regions and (ii) classify the given data point as not having the particular property when the confidence score does not satisfy the operating point and is not within any of the one or more deferral regions. The terms “satisfy the operating point” is used here to mean “satisfy a criterion comparing the confidence score to the operating point”.

That is, upon determining that the confidence score for the given data point does not fall within any of the one or more deferral regions, the model determines whether the confidence score satisfies the operating point.

When higher confidence scores indicate a greater likelihood of a data point having the property, the confidence score satisfies the operating point when the confidence score is greater than the operating point and does not satisfy the operating point when the confidence is less than or equal to the operating point.

When lower confidence scores indicate a greater likelihood of a data point having the property, the confidence score satisfies the operating point when the confidence score is less than the operating point and does not satisfy the operating point when the confidence is greater than or equal to the operating point.

When the confidence score satisfies the operating point, the model determines to classify the given data point as having the particular property.

When the confidence score does not satisfy the operating point, the model determines to classify the given data point as not having the particular property.

Thus, by determining the respective lower and upper bounds for each of the one or more deferral regions and the operating point, the training system 190 determines how the deferral model will operate after training.

In other words, determining the parameters of the deferral model 110 requires determining the respective lower and upper bounds for each of the one or more deferral regions and the operating point using the tuning data set 180.

FIG. 3 shows an example of the composition of the tuning data set 180. The operations to generate the tuning data set 180 can be performed by the training system 190 or by an external system and provided as input to the training system 190.

In the example of FIG. 3, like in the example of FIG. 2, the input data points are medical cases for corresponding patients and the classification output indicates whether a specified disease is present or absent in the input medical case.

Thus, the tuning data set includes tuning medical cases 302. Each tuning medical case 302 is processed using the already trained diagnostic model(s) 120 to generate a respective confidence score 304 for each tuning medical case 302. During this process, the weights, i.e., the parameters, of the model(s) 120 are “frozen,” meaning that the weights are held constant to their trained values and the model(s) 120 are not trained further.

Each tuning medical case 302 is also provided through the clinical workflow 210 to obtain a user indication (“retrospective clinician opinion) 306 for each tuning medical case 302 that identifies whether the one or more clinicians evaluated the tuning medical case 302 as having the particular property or as not having the particular property, i.e., whether the one or more clinicians indicated that the disease was present or absent in the tuning medical case 302.

Additionally, each tuning medical case 302 is associated with a label 308 (also included in the tuning data set 108) that identifies a “ground truth” classification for the tuning medical case 302 as either a positive tuning data point or a negative tuning data point. In the example of FIG. 3, the label has been generated as a result of testing, i.e., a biopsy, being performed on the patient characterized by the tuning medical case 302.

The training system 190 then uses the tuning data set 180 to perform a training process 310 to determine the parameters of the deferral model 110.

FIG. 4 is a flow diagram of an example process 400 for performing a training process to determine the parameters of the deferral model. For convenience, the process 400 will be described as being performed by a system of one or more computers located in one or more locations. For example, a training system, e.g., the training system 100 of FIG. 1, appropriately programmed, can perform the process 400.

The system obtains a tuning data set for learning the parameters of the deferral model (step 402). As described above, the tuning data set includes, for each of a plurality of positive tuning data points and a plurality of negative data points, (i) a confidence score generated by the set of trained diagnostic machine learning models for the tuning data point and (ii) a user indication for the tuning data point that identifies whether a user evaluated the tuning data point as having the particular property or as not having the particular property.

Thus, the tuning data set can be expressed as:

$${D}_{tune} = ( {z}^{i} , {h}^{i} , {y}^{i} ) , i = 1 , … , N$$

where N is the total number of data points hⁱ is the user indication for data point i, yⁱ the ground truth label that indicates whether the data point i is a positive or negative data point and z the confidence score for the data point i.

The system determines the parameters of the deferral model by optimizing an objective that is based on a specificity of the deferral model on the tuning data set and a sensitivity of the deferral model on the tuning data set (step 404).

In particular, for each of k deferral regions, the system determines the lower bound αₖ and the upper bound βₖ for the deferral regions such that:

$$0 < {α}_{1} < {β}_{1} < {α}_{2} < {β}_{2} … < {α}_{k} < {β}_{k} <= 1$$

The system also determines an operating point θ that is between zero and one (e.g. when each confidence score zⁱ is between zero and one).

For example, the system can determine parameters that maximize a weighted sum of (i) the sensitivity and (ii) the specificity of the deferral model. The weight in the weighted sum specifies a desired trade-off between sensitivity and the specificity.

The weight can be received as input or can be determined using a hyperparameter search by the system, as will be described in more detail below.

In some cases, the system selects the operating point θ and then determines the lower and upper bound of the deferral region(s) that will maximize the objective given the selected operating point θ.

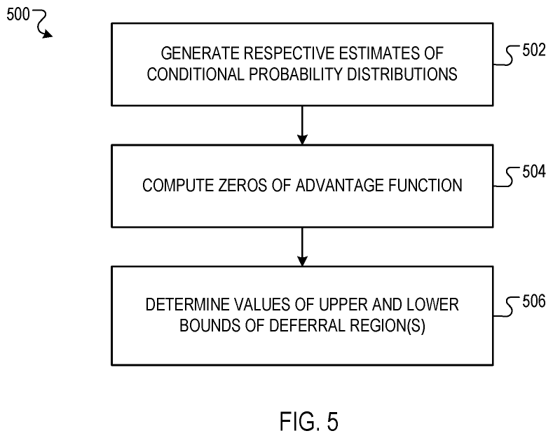

An example of such a technique is described below with reference to FIG. 5.

As another example, the system can determine the lower and upper bound of the deferral region(s) and the operating point jointly, e.g. through dynamic programming.

For example, the system can determine the parameters using dynamic programming by performing a constrained optimization of an objective that maximizes the weighted sum of the specificity and the sensitivity subject to one or more constraints that ensure that the result of the optimization results in valid parameters.

For example, the system can assign each tuning data point a respective tuning data point index based on a position of the confidence score for the tuning data point in a ranking of the confidence scores for the tuning data points.

The one or more constraints can then include a first constraint on the operating point that specifies that there must be at most one tuning data point index for which (i) all tuning data points with index less than the tuning data point index have a confidence score that does not satisfy the operating point and (ii) all tuning data points with index greater than or equal to the tuning data point index have a confidence score that does satisfy the operating point.

As another example, the one or more constraints can alternatively or additionally include a second constraint that specifies that a number of tuning data point indices for which the deferral decision is different between (i) the tuning data point having the tuning data point index and (ii) the tuning data point having an index that is one higher than the tuning data point index does not exceed a maximum threshold value. As a result of this constraint, if the maximum threshold value is 2k, there will be at most k deferral regions.

Then, the system determines, through dynamic programming, an optimal solution to a dynamic programming formulation of the constrained maximization and then determines the parameters from the optimal solution.

After determining the parameters, the system or another system uses the deferral model to determine whether to “defer” new data points to a user as described above (step 406).

In some cases, the system uses deferral model to adapt the system from one domain to another without needing to re-train the underlying diagnostic machine learning models, resulting in significant resource savings.

In particular, the diagnostic machine learning models may have been trained on input data points that have a first distribution but may then need to be deployed to classify downstream data points that are distributed differently. For example, in the medical imaging application, the distribution of medical images may be different during training than when the system is deployed.

In this case, the system can obtain a tuning data set that is drawn from the distribution of the downstream data points, i.e., from the distribution of data points that the system will be required to classify after training, and adapt the AI system to function well on the downstream data points without needing to re-train the underlying model(s) by learning the parameters of the deferral model using the tuning data set.

FIG. 5 is a flow diagram of an example process 500 for determining the upper and lower bounds of the deferral region(s) using the tuning data set. For convenience, the process 500 will be described as being performed by a system of one or more computers located in one or more locations. For example, a training system, e.g., the training system 100 of FIG. 1, appropriately programmed, can perform the process 500.

As described above, prior to performing the process 500, the system selects a value for the operating point. For example, the system can treat the operating point as a hyperparameter and perform a hyperparameter search for the “optimal” value of the operating point. As part of this search, the system can sample multiple candidate values for the operating point and perform the process 500 for each candidate value. The system can then select the operating point that results in the best performing deferral model as the final operating point. For example, the best performing model can be the one that most improves on a baseline measure, e.g., classifying using only the diagnostic models or only using user feedback, on a validation set that is separate from the tuning data set.

The system generates a respective estimate of each of a set of conditional probability distributions using the tuning data set (step 502). Each conditional probability distribution corresponds to a respective user indication and a respective label and, for each possible confidence score value, assigns a respective probability to a test case having the possible confidence score value and the corresponding user indication given that the test case has the corresponding label.

In particular, the system generates estimates of the following conditional probability distributions:
(i) P(z, h=1|y=0), i.e., for z between zero and one, the probability that the classification score for a data point is equal to z and the user indication indicates that the data point has the particular property given that the ground truth label for the data point is negative,
(ii) P(z, h=0|y=0), i.e., for z between zero and one, the probability that the classification score for a data point is equal to z and the user indication indicates that the data point does not have the particular property given that the ground truth label for the data point is negative,
(iii) P(z, h=1|y=1), i.e., for z between zero and one, the probability that the classification score for a data point is equal to z and the user indication indicates that the data point has the particular property given that the ground truth label for the data point is positive, and
(iv) P(z, h=0|y=1), i.e., for z between zero and one, the probability that the classification score for a data point is equal to z and the user indication indicates that the data point does not have the particular property given that the ground truth label for the data point is positive.

The system can estimate these conditional probability distributions by applying any appropriate density estimation technique.

One example of such a technique is kernel density estimation.

A specific example of an approach for applying a density estimation technique to estimate any of the four conditional probability distributions P(z, h=a|y=b) where a, b∈{0, 1}now follows.

The system divides the interval [0, 1] into N_{b} bins of equal width

$${I}_{t} = [ \frac{t - 1}{{N}_{b}} , \frac{t}{{N}_{b}} ]$$

with t=1, . . . , N_{b}. For each bin t, the system then counts the number of data points in Dₜᵤₙₑ such that

y=b, h=a, z∈Iₜ

This count can be denoted as c_{y,h,t}. The system additionally adds a smoothing parameter κ>0 to the counts (so as to avoid assigning 0 counts to certain bins even when data is highly sparse).

The system defines an M-component Gaussian RBF kernel by, for the j-th component, with center parameter ξᵢ=(j−½)/M) and spread a as

$${R}_{j} ( x ) = \frac{exp ( - {( x - {ξ}_{j} )}^{2} / 2 {σ}^{2}}{{\sum }_{k} exp ( - {( x - {ξ}_{k} )}^{2} / 2 {σ}^{2} )}$$

Let μₜ be the midpoint of Iₜ. Then, the kernels at μₜ evaluate to

B_{t,j}=Rⱼ(μₜ)

The system models the smoothed counts as:

$${c}_{y , h , t} + κ \approx {c}_{y , h , t}^{est} ( w ) = {\sum }_{j = 1}^{M} {B}_{t , j} {w}_{j , y , h}$$

and optimizes w by solving

$$minimize {\sum }_{y , h , t} KL ( {c}_{y , h , t} + κ ‖ {c}_{y , h , t}^{est} ( w ) )$$

$$where$$

$$KL ( u ‖ v ) = v log ( u / v ) - u + v$$

denotes the Kullback-Leibler divergence between un-normalized distributions represented by arbitrary positive real numbers u, v.

The system can solve for w using an iterative minimization procedure using multiplicative updates similar to non-negative matrix factorization, where only w_{j,y,h} are updated by keeping B_{t,j} fixed.

Optionally, instead of performing the hyperparameter search only over values of the operating points, the system can also treat the values required by the density estimation technique as hyperparameters and involve them in the hyperparameter search. For example, the system can treat all of θ, λ, κ, N_{b}, M as hyperparameters and perform a sweep over multiple combinations of possible values for each and then select the best performing resulting model as described above.

Once the system has generated the respective estimate of each of the set of conditional probability distributions, the system computes the zeros of an advantage function given that the operating value is set to the selected value (step 504). That is, the system determines all confidence scores z between zero and one for which the advantage function is equal to zero.

The advantage function is a function of the confidence score z that, given the conditional probability distribution estimates and the operating point, is greater than zero when deferring to the user is expected to result in a higher objective value for a data point having the confidence score z and is less than zero when classifying the data point using the confidence score z is expected to result in a higher objective value for the data point.

More specifically, the advantage function is a function of the confidence scores z and is defined as:

$$minimize {\sum }_{y , h , t} KL ( {c}_{y , h , t} + κ ‖ {c}_{y , h , t}^{est} ( w ) )$$

$$where$$

$$KL ( u ‖ v ) = v log ( u / v ) - u + v$$

where λ is the weight for the sensitivity and 1−λ is the weight for the specificity in the objective function.

The set of zeros, when ordered in ascending order, can be denoted as 0≤p₁<p₂< pₖ≤1 where each pᵢ is a value of z for which Advantage(z) is equal to zero and k is the total number of zeros.

The system determines the values of the upper and lower bounds of the deferral region(s) using the zeros (step 506).

In particular, the system can compute the deferral thresholds as follows:
a. If Advantage(0)≥0 and k is even set α₀=0, β₀=p₁, α₁=p₂, . . . α_{k/2}=pₖ, β_{k/2}=1.
b. If Advantage(0)≥0 and k is odd, set α₀=0, β₀=p₁, α₁=p₂, . . . , α_{(k-1)/2}=pₖ₋₁, β_{(k-1)/2}=pₖ.
c. If Advantage(0)<0 and k is odd, set α₀=p₁, β₀=p₂, α₁=p₃, . . . α_{(k-1)/2}=pₖ, β_{(k-1)/2}=1.
d. If Advantage(0)<0 and k is even, set α₀=p₁, β₀=p₂, α₁=p₃, . . . , α_{k/2-1}=pₖ₋₁, β_{k/2-1}=pₖ.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a data communication network.

In this specification, the term “database” is used broadly to refer to any collection of data: the data does not need to be structured in any particular way, or structured at all, and it can be stored on storage devices in one or more locations. Thus, for example, the index database can include multiple collections of data, each of which may be organized and accessed differently.

Similarly, in this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework or a Jax framework.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method performed by one or more computers, the method comprising:
obtaining a tuning data set for learning parameters of a deferral model, wherein:
the deferral model is configured to:
receive as input a confidence score generated by a set of one or more trained diagnostic machine learning models for a given data point that represents an estimated probability that the given data point has a particular property and
determine, based on the parameters, whether to (i) classify the given data point as having the particular property, (ii) classify the given data point as not having the particular property, or (iii) provide the given data point for presentation to a user for evaluation of whether the given data point has the particular property, and
the tuning data set comprises, for each of a set of tuning data points that comprises a plurality of positive tuning data points that have been labeled as having the particular property and a plurality of negative data points that have been labeled as not having the particular property, (i) a confidence score generated by the set of trained diagnostic machine learning models for the tuning data point and (ii) a user indication for the tuning data point that identifies whether a user evaluated the tuning data point as having the particular property or as not having the particular property; and
determining the parameters of the deferral model by optimizing an objective that is based on a specificity of the deferral model on the tuning data set and a sensitivity of the deferral model on the tuning data set, wherein:
the specificity of the deferral model measures a first fraction of the positive tuning data points for which either (i) the deferral model determined to classify the positive tuning data point as having the particular property or (ii) the deferral model determined to provide the positive tuning data point for evaluation by a user and the user indication for the positive tuning data point identifies that the user evaluated the positive tuning data point as having the particular property, and
the sensitivity of the deferral model measures a second fraction of the negative tuning data points for which either (i) the deferral model determined to classify the negative tuning data point as not having the particular property or (ii) the deferral model determined to provide the negative tuning data point for evaluation by a user and the user indication for the negative tuning data point identifies that the user evaluated the negative tuning data point as not having the particular property.

2. The method of claim 1, wherein the given data point comprises one or more medical images for a patient, the particular property relates to a medical condition, and the user is a clinician.

3. The method of claim 1, wherein determining the parameters of the deferral model by optimizing the objective comprises determining the parameters of the deferral model without further training the trained diagnostic machine learning models.

4. The method of claim 1, wherein the set of one or more trained diagnostic machine learning models comprises a plurality of trained diagnostic machine learning models and wherein the confidence score for the given data point is a weighted combination of model outputs generated by each of the trained diagnostic machine learning models by processing the given data point.

5. The method of claim 1, wherein the parameters of the deferral model define:
respective lower and upper bounds for each of one or more deferral regions, wherein the deferral model determines to provide the given data point for presentation to a user for evaluation when the confidence score for the given data point falls within any of the one or more deferral regions, and
an operating point, wherein the deferral model determines to:
classify the given data point as having the particular property when the confidence score satisfies the operating point and is not within any of the one or more deferral regions, and
classify the given data point as not having the particular property when the confidence score does not satisfy the operating point and is not within any of the one or more deferral regions.

6. The method of claim 5, wherein determining the parameters of the deferral model comprises:
determining parameters that maximize a constrained maximization of a weighted sum of the sensitivity and the specificity of the deferral model on the tuning data set subject to one or more constraints.

7. The method of claim 6, wherein each tuning data point is assigned a respective tuning data point index based on a position of the confidence score for the tuning data point in a ranking of the confidence scores for the tuning data points, and wherein the one or more constraints include a first constraint on the operating point that specifies that there must be at most one tuning data point index for which (i) all tuning data points with index less than the tuning data point index have a confidence score that does not satisfy the operating point and (ii) all tuning data points with index greater than or equal to the tuning data point index have a confidence score that does satisfy the operating point.

8. The method of claim 6, wherein each tuning data point is assigned a respective tuning data point index based on a position of the confidence score for the tuning data point in a ranking of the confidence scores for the tuning data points, and wherein the one or more constraints include a second constraint that specifies that a number of tuning data point indices for which the deferral decision is different between (i) the tuning data point having the tuning data point index and (ii) the tuning data point having an index that is one higher than the tuning data point index does not exceed a maximum threshold value.

9. The method of claim 6, wherein determining parameters that maximize the weighted sum subject to the one or more constraints comprises:
determining, through dynamic programming, an optimal solution to a dynamic programming formulation of the constrained maximization; and
determining the parameters from the optimal solution.

10. The method of claim 5, wherein determining the parameters of the deferral model comprises:
determining parameters that maximize an objective that measures a weighted sum of the sensitivity and the specificity of the deferral model on the tuning data set.

11. The method of claim 10, wherein determining parameters that maximize the objective comprises:
selecting a value for the operating point;
determining, using the tuning data set, a respective estimate for each of a set of conditional probability distributions that each correspond to a respective user indication and a respective label and, for each possible confidence score value, assign a respective probability to a test case having the possible confidence score value and the corresponding user indication given that the test case has the corresponding label;
determining zeros of an advantage function using the respective estimates and given that the operating point is set to the selected value; and
determining the respective upper and lower bounds for each of the one or more deferral regions using the zeros of the advantage function.

12. The method of claim 11, wherein determining a respective estimate for each of a set of conditional probability distributions comprises applying density estimation to the tuning data set.

13. The method of claim 11, wherein the advantage function is a function of a confidence score z that, given the respective conditional probability distribution estimates and the selected value of the operating point, is greater than zero when deferring is expected to result in a higher objective value for a data point having the confidence score z and is less than zero when classifying the data point using the confidence score z is expected to result in a higher objective value for the data point.

14. A method comprising:
receiving a new data point;
processing the new data point using one or more trained diagnostic machine learning models to generate a new confidence score for the new data point that represents an estimated likelihood that the new data point has a particular property; and
processing the new data point using a deferral model having parameters to determine whether to (i) classify the new data point as having the particular property, (ii) classify the new data point as not having the particular property, or (iii) provide the new data point for presentation to a user for evaluation of whether the given data point has the particular property, wherein the parameters of the deferral model have been learned by performing operations comprising:
obtaining a tuning data set for learning parameters of the deferral model, wherein:
the tuning data set comprises, for each of a set of tuning data points that comprises a plurality of positive tuning data points that have been labeled as having the particular property and a plurality of negative data points that have been labeled as not having the particular property, (i) a confidence score generated by the set of trained diagnostic machine learning models for the tuning data point and (ii) a user indication for the tuning data point that identifies whether a user evaluated the tuning data point as having the particular property or as not having the particular property; and
determining the parameters of the deferral model by optimizing an objective that is based on a specificity of the deferral model on the tuning data set and a sensitivity of the deferral model on the tuning data set, wherein:
the specificity of the deferral model measures a first fraction of the positive tuning data points for which either (i) the deferral model determined to classify the positive tuning data point as having the particular property or (ii) the deferral model determined to provide the positive tuning data point for evaluation by a user and the user indication for the positive tuning data point identifies that the user evaluated the positive tuning data point as having the particular property, and
the sensitivity of the deferral model measures a second fraction of the negative tuning data points for which either (i) the deferral model determined to classify the negative tuning data point as not having the particular property or (ii) the deferral model determined to provide the negative tuning data point for evaluation by a user and the user indication for the negative tuning data point identifies that the user evaluated the negative tuning data point as not having the particular property.

15. The method of claim 14, further comprising:
determining to classify the new data point as having the particular property; and
in response, providing, as output, an indication that the new data point has the particular property and, optionally, the new confidence score for the new data point.

16. The method of claim 14, further comprising:
determining to classify the new data point as not having the particular property; and
in response, providing, as output, an indication that the new data point does not have the particular property and, optionally, the new confidence score for the new data point.

17. The method of claim 14, further comprising:
determining to provide the new data point for presentation to a user for evaluation of whether the given data point has the particular property; and
in response, providing the new data point for presentation on a user computer.

18. A system comprising one or more computers and one or more storage devices storing instructions that when executed by the one or more computers cause the one more computers to perform operations comprising:
obtaining a tuning data set for learning parameters of a deferral model, wherein:
the deferral model is configured to:
receive as input a confidence score generated by a set of one or more trained diagnostic machine learning models for a given data point that represents an estimated probability that the given data point has a particular property and
determine, based on the parameters, whether to (i) classify the given data point as having the particular property, (ii) classify the given data point as not having the particular property, or (iii) provide the given data point for presentation to a user for evaluation of whether the given data point has the particular property, and
the tuning data set comprises, for each of a set of tuning data points that comprises a plurality of positive tuning data points that have been labeled as having the particular property and a plurality of negative data points that have been labeled as not having the particular property, (i) a confidence score generated by the set of trained diagnostic machine learning models for the tuning data point and (ii) a user indication for the tuning data point that identifies whether a user evaluated the tuning data point as having the particular property or as not having the particular property; and
determining the parameters of the deferral model by optimizing an objective that is based on a specificity of the deferral model on the tuning data set and a sensitivity of the deferral model on the tuning data set, wherein:
the specificity of the deferral model measures a first fraction of the positive tuning data points for which either (i) the deferral model determined to classify the positive tuning data point as having the particular property or (ii) the deferral model determined to provide the positive tuning data point for evaluation by a user and the user indication for the positive tuning data point identifies that the user evaluated the positive tuning data point as having the particular property, and
the sensitivity of the deferral model measures a second fraction of the negative tuning data points for which either (i) the deferral model determined to classify the negative tuning data point as not having the particular property or (ii) the deferral model determined to provide the negative tuning data point for evaluation by a user and the user indication for the negative tuning data point identifies that the user evaluated the negative tuning data point as not having the particular property.

19. One or more non-transitory computer storage media storing instructions that when executed by one or more computers cause the one more computers to perform operations comprising:
obtaining a tuning data set for learning parameters of a deferral model, wherein:
the deferral model is configured to:
receive as input a confidence score generated by a set of one or more trained diagnostic machine learning models for a given data point that represents an estimated probability that the given data point has a particular property and
determine, based on the parameters, whether to (i) classify the given data point as having the particular property, (ii) classify the given data point as not having the particular property, or (iii) provide the given data point for presentation to a user for evaluation of whether the given data point has the particular property, and
the tuning data set comprises, for each of a set of tuning data points that comprises a plurality of positive tuning data points that have been labeled as having the particular property and a plurality of negative data points that have been labeled as not having the particular property, (i) a confidence score generated by the set of trained diagnostic machine learning models for the tuning data point and (ii) a user indication for the tuning data point that identifies whether a user evaluated the tuning data point as having the particular property or as not having the particular property; and
determining the parameters of the deferral model by optimizing an objective that is based on a specificity of the deferral model on the tuning data set and a sensitivity of the deferral model on the tuning data set, wherein:
the specificity of the deferral model measures a first fraction of the positive tuning data points for which either (i) the deferral model determined to classify the positive tuning data point as having the particular property or (ii) the deferral model determined to provide the positive tuning data point for evaluation by a user and the user indication for the positive tuning data point identifies that the user evaluated the positive tuning data point as having the particular property, and
the sensitivity of the deferral model measures a second fraction of the negative tuning data points for which either (i) the deferral model determined to classify the negative tuning data point as not having the particular property or (ii) the deferral model determined to provide the negative tuning data point for evaluation by a user and the user indication for the negative tuning data point identifies that the user evaluated the negative tuning data point as not having the particular property.

20. The system of claim 18, wherein the given data point comprises one or more medical images for a patient, the particular property relates to a medical condition, and the user is a clinician.
